(19) United States
(12) Patent Application Publication
(10) Pub. No.: US 2005/0118565 A1
(43) Pub. Date: Jun. 2, 2005

(54) Systems and methods for assessing neuronal degeneration

(76) Inventors: Ken Shimono, Hirakata (JP); Victor Panchenko, San Diego, CA (US); Makoto Taketani, Irvine, CA (US); Michael Krause, Irvine, CA (US)

Correspondence Address: MORRISON & FOERSTER LLP, 755 PAGE MILL RD, PALO ALTO, CA, 94304-1018, US

(21) Appl. No.: 10/966,405

(22) Filed: Oct. 15, 2004

Related U.S. Application Data
(60) Provisional application No. 60/511,948, filed on Oct. 15, 2003.

(51) Int. Cl. C12Q 1/00; A61K 31/55; A61K 31/42

(52) U.S. Cl. 435/4; 514/458; 514/217.01; 514/378; 205/777.5; 514/561

(57) ABSTRACT

The invention relates to systems and methods for assessing neuronal degeneration in vitro. A multielectrode probe is used to culture and measure synaptic transmission of a neuronal sample in the presence of various compounds and culturing conditions capable of inducing or protecting against neuronal damage.

### CROSS-REFERENCE TO RELATED APPLICATIONS

This application claims priority to U.S. Provisional Application Ser. No. 60/511,948, filed on Oct. 15, 2003, the disclosure of which is herein incorporated by reference in its entirety.

### FIELD

Described here are systems and methods for the in vitro assessment of neurodegeneration. More specifically, systems and methods for assessing whether selected compounds induce or protect against neurodegeneration are described. The systems and methods may also be used to assess whether selected culturing conditions induce a neurodegenerative or neuroprotective effect.

### BACKGROUND

In vitro studies of the mechanisms of neuronal cell death, for example, neuronal degeneration secondary to excitotoxicity, have traditionally been performed in primary neuronal cultures or in organotypic cultures of hippocampal slices using fluorescent markers that detect various aspects of neuronal cell integrity (Vornov J. J. and Coyle J. T., J. Neurochem. 56:996-1006 (1991); Choi D. W., J. Neurobiol., 23:1261-1276 (1992); and Bruce et al., Exp. Neurol. 132:209-219 (1995)). These methods typically use a marker that binds a target molecule, such as a gene or protein, or a marker that binds cell membranes. These conventional methods do not measure changes in the activity of neurons per se, but rather, measure changes in the molecules and/or cells affected by the death of neurons. In general, these methods are easy to implement and are amenable to large scale screening of potential neuroprotective compounds. However, they do not provide a detailed time-course of events leading to cell death, nor do they discriminate the fate of different populations of neurons (or of glial cells).

In principle, electrophysiological techniques are better suited to provide information about neuronal degeneration. However, until recently, commercially available electrophysiologic recording devices were limited to recording the activity of only one or a few neurons. Furthermore, they were only able to record, at most, 6-10 hours of electrophysiological activity.

Planar electrode arrays have now been developed that simultaneously record the activity of multiple neurons in neuronal cultures (Pine J., J. Neurosci. Methods, 2:19-31 (1980) and Gross et al., J. Neurosci. Methods, 5:13-22 (1982)), organotypic cultures (Stoppini et al., J. Neurosci. Methods, 72:23-33 (1997) and Egert et al., Brain Res. Protoc., 2:229-242 (1998)), and acute slices (Novak J. L. and Wheeler B. C., J. Neurosci. Methods, 23:149-159 (1998); Oka et al., J. Neurosci. Methods, 93:61-67 (1999); and Gholmieh et al., Biosens Bioelectron, 16:491-501 (2001)). A device that records from as many as 64 electrodes uniformly distributed in a brain slice has also been described in U.S. Pat. No. 6,297,025 and U.S. Pat. No. 6,132,683 to Sugihara et al. which are herein incorporated by reference in their entirety. In a typical hippocampal slice, these arrays can monitor the electrophysiological status of neurons in all hippocampal subfields (Stoppini et al., J. Neurosci. Methods, 72:23-33 (1997) and Oka et al., J. Neurosci. Methods, 93:61-67 (1999)). However, none of these documents describe a method for studying the chronic effect of compounds or of other experimental manipulations on the electrical activity of injured or dying neurons.

Neurodegeneration has been implicated in the pathophysiology of such conditions as Huntington's Disease, Parkinson's Disease, Alzheimer's Disease, vascular dementia, amyotrophic lateral sclerosis, ischemia, and Down's Syndrome. The etiology of neuronal damage for these conditions has been attributed to such pathologic mechanisms as excitotoxic neuronal damage (excitotoxicity) or oxidative damage. Therefore, systems and methods for assessing and preventing neuronal damage may provide significant medical benefit.

### SUMMARY OF THE INVENTION

The invention generally provides systems and methods for the assessment of neurodegeneration. Specifically, systems and methods for assessing whether candidate compounds or culturing conditions induce neuronal damage, or protect against neuronal damage are described.

In one variation, the method for assessing whether a selected compound induces neurodegeneration in vitro includes: 1) providing a device having a plurality of microelectrodes on a substrate configured to contact a neuronal sample and apply an electric stimulus to the neuronal sample; 2) contacting the neuronal sample with the plurality of microelectrodes; 3) measuring a baseline synaptic transmission of the neuronal sample; 4) contacting the neuronal sample with a first candidate compound; 5) measuring a first resultant synaptic transmission of the neuronal sample at one or more timepoints after contacting the neuronal sample with the first candidate compound; and 6) comparing the first resultant synaptic transmission with the baseline synaptic transmission. A decrease in synaptic transmission between the first and baseline synaptic transmissions generally indicates that the candidate compound induces neurodegeneration in the neuronal sample.

Alternatively, this procedure may be used to determine whether candidate compounds protect against neuronal damage by performing steps 1-5, as described above, in addition to: contacting the neuronal sample with a second candidate compound; measuring a second resultant synaptic transmission of the neuronal sample at one or more timepoints after contacting the neuronal sample with the second candidate compound; and comparing the second resultant synaptic transmission with the first synaptic transmission. An increase in synaptic transmission between the second and first synaptic transmissions generally indicates that the second candidate compound provides some measure of protection against neurodegeneration in the neuronal sample.

The systems and methods described here may be used to detect or assess a culturing condition that induces neuronal damage. The method may include: 1) providing a device having a plurality of microelectrodes on a substrate configured to contact a neuronal sample and apply an electric stimulus to the neuronal sample, and a culturing chamber that provides a first culturing condition; 2) contacting the neuronal sample with the plurality of microelectrodes; 3) measuring a baseline synaptic transmission of the neuronal sample; 4) altering the first culturing condition to produce a second culturing condition; 5) measuring a first synaptic transmission of the neuronal sample at one or more timepoints after altering the first culturing condition to produce a second culturing condition; and 6) comparing the first synaptic transmission with the baseline synaptic transmission. A decrease in synaptic transmission between the first and baseline synaptic transmissions generally indicates that the second culturing condition induces neurodegeneration in the neuronal sample.

In a further variation, culturing conditions resulting in at least partial protection against neuronal damage may be assessed by performing steps 1-5, as described above, in addition to: altering the second culturing condition; measuring a second resultant synaptic transmission of the neuronal sample at one or more timepoints after altering the second culturing condition; and comparing the second resultant synaptic transmission with the first synaptic transmission. An increase in synaptic transmission between the second and first synaptic transmissions generally indicates that the altered second culturing condition protects against neurodegeneration in the neuronal sample.

### BRIEF DESCRIPTION OF THE DRAWINGS

FIG. 1A is a digital image of a hippocampal sample being cultured on a MED probe.

FIG. 1B shows electrical waveforms obtained using a paired-pulse stimulation paradigm.

FIG. 1C shows a timeline of amplitude measurements of electrical waveforms taken in screening assays for candidate compounds.

FIG. 2A shows electrical waveforms obtained after stimulation at various current intensities.

FIG. 2B is an input-output (I/O) curve demonstrating peak amplitudes of electrical waveforms as a function of stimulation intensity.

FIG. 2C compares the slope and decay times of electrical waveforms obtained from neural tissue slices in acute and long-term cultures.

FIG. 2D is the I/O curve obtained for the experimental control (acute hippocampal sample).

FIG. 3A is an I/O curve that shows the reduction in amplitude of electrical waveforms after exposure to NMDA.

FIG. 3B is an I/O curve that shows the reduction in amplitude of electrical waveforms after exposure to AMPA.

FIG. 3C shows representative electrical waveforms from FIG. 3A.

FIG. 3D shows representative electrical waveforms from FIG. 3B.

FIG. 4A shows the effect of increasing NMDA concentration on the amplitude of electrical waveforms.

FIG. 4B shows the effect of increasing AMPA concentration on the amplitude of electrical waveforms.

FIG. 5 shows the amount of recovery of synaptic responses at various timepoints after NMDA removal.

FIG. 6A is an I/O curve that shows the protective effect of MK-801 on neuronal tissue incubated with NMDA.

FIG. 6B is an I/O curve that demonstrates the protective effect of memantine on neuronal tissue incubated with NMDA.

FIG. 6C shows the direct correlation between memantine concentration and degree of neuroprotection.

FIG. 7A shows the effect of MK-801 on synaptic responses.

FIG. 7B shows the effect of memantine on synaptic responses.

FIG. 8 demonstrates the effect of chronic exposure to NMDA receptor agonists and/or antagonists on synaptic responses.

### DETAILED DESCRIPTION OF THE INVENTION

Described herein are systems and methods for assessing neuronal degeneration in vitro. By “neuronal degeneration” or “neuronal damage” it is meant a change in synaptic transmission between neurons, often an injury or death of one or more neurons such that synaptic transmission between neurons is decreased. The systems and methods may use a multielectrode device, further described below, to measure synaptic transmission activity of the neuronal samples. As used herein, the term “neuronal sample” refers, in context, variously to individual neurons, aggregations of neural cells, one or more layers of neural cells, and neural tissue slices. Specifically, where a parameter of some type is to be measured, e.g., an electrical waveform, the “context” may be that the “neuronal sample” be of a size sufficient to generate a measurable parameter value. One way in which the multielectrode device measures synaptic transmission is by measuring the amplitude of electrical waveforms generated by a specific portion of the neuronal sample. For instance, a decrease in the amplitude of an electrical waveform typically indicates a decrease in synaptic transmission and an increase in the amplitude of an electrical waveform typically indicates an increase in synaptic transmission.

Multielectrode Device for Assessing Neuronal Degeneration

The systems and methods described here may use a device that includes a number of cell potential measuring electrodes previously described in U.S. Pat. No. 6,132,683 to Sugihara et al. to obtain and to record the electrical activity of neurons and neuronal samples. Sugihara et al.'s device, in all of its published variations, will be generally referred to as a MED probe. Other devices having multiple electrode sites suitable for measuring the neuronal electrical activity discussed below and, to the extent needed in the described systems and procedures, to provide electrical excitation in a specified fashion, will also be suitable for practice of the described systems and procedures. In certain instances, we may refer to the use of a MED probe where other functionally similar or otherwise appropriate devices may be selected.

In brief, the MED probe includes a plurality of physically isolated microelectrodes perhaps on an insulating substrate, and generally having a conductive pattern for connecting the microelectrodes to some region out of the microelectrode area. As a practical matter, the device may also have electric contacts connected to the end of the conductive pattern, an insulating film covering the surface of the conductive pattern, and a wall enclosing the region including the microelectrodes on the surface of the insulating film. In some variations of the MED, reference electrodes having a comparatively lower impedance than the impedance of the measuring microelectrodes are placed at plural positions in the region enclosed by the wall and often at a specific distance from the microelectrodes. The electrical contacts are further usually connected between the conductive pattern for wiring of each reference electrode and the end of the conductive pattern. The surface of the conductive pattern for wiring of the reference electrodes is typically covered with an insulating film. In addition, many variations of the MED probe include an optical observation device, e.g., an inverted microscope, for optically measuring or observing the neuronal sample placed on the probe, a computer for giving a stimulus signal to the sample and for processing the output signal from the sample, and a culturing chamber for maintaining a culture atmosphere about the sample.

The computer is typically a personal computer (PC) in which measurement/stimulus software is installed. The computer and multielectrode device are overall connected through an I/O board for measurement. The I/O board typically includes both A/D converter and D/A converters. The A/D converter is usually for measuring and converting the resulting potentials; the D/A converter is for sending stimulus signals to the sample. For example, the A/D converter may have 16 bits, 64 channels, and the D/A converter may have 16 bits, 8 channels. In this instance, software able to determine the time-wise variation of synaptic transmission during the procedure may be installed on the PC.

One typical procedure for providing a stimulus to the sample is this: when a stimulus signal is issued from the computer, the stimulus signal is sent to the multielectrode device through the D/A converter and an isolator. The induced, evoked, or spontaneous potential occurring between each microelectrode and reference potential is passed through a 64 channel high sensitivity amplifier and the A/D converter into the computer. The amplification factor of the amplifier may be, e.g., about 80-100 dB, for example, in a frequency band of about 0.1 to 10 kHz, or to 20 kHz. However, when measuring the potential induced by a stimulus signal, by using a low-cut filter, the frequency band is 100 Hz to 10 kHz. Spontaneous potentials are usually in the range of 100 Hz to 20 Hz.

For data analysis or processing, Fourier Function Transform (FFT) analysis, coherence analysis, and correlation analysis may be employed. Useable functions may include single spike separation functions using waveform discrimination, a temporal profile display functions, topography display functions, and current source density analysis functions.

A culturing chamber may be a part of a culturing system that generally includes a temperature controller, a culture fluid circulation device, and a feeder for supplying, e.g., a mixed gas of air and carbon dioxide. The culturing system may instead be made up of a commercial microincubator, a temperature controller, and CO₂ cylinder. The microincubator can be used to control the temperature in a range of 0° C. to 50° C. by use of a Peltier element and is applicable to the liquid feed rate of 3 ml/min or less and gas flow rate of 1 L/min or less. Or, a microincubator incorporating other designs of temperature controllers may be used.

Methods for Assessing Neuronal Degeneration

Typically, the methods for assessing neuronal degeneration involve MED probe and neuronal sample preparation, and recording of baseline electrical waveforms as described in Examples 1, 2, and 3 respectively. Neuronal samples may be taken from any area of the brain or spinal cord, including, but not limited to, the hippocampus, the substantia nigra, the cerebellum, the thalamus, the hypothalamus, and the like.

We use the terms “detecting” and “assessing” in a number of ways. The procedures described here will be used to “detect” the absence or presence of a compound and also to “detect” the relative efficacy of a compound in terms of its making a change to a parameter of the neuronal sample that is measurable using a device or procedure capable of measuring the affected parameter. The term “assessing” is used in the same way.

In one variation, the method detects compounds that are capable of inducing neuronal damage. After baseline synaptic transmission is recorded, a candidate compound is added to the culture medium such that it contacts the sample. Synaptic transmission is then recorded at various timepoints, e.g., at 3 hours, 1 day, 2 days, or 3 days or more, after introduction of the candidate compound, depending on investigator preference. Decreases in synaptic transmission observed when compared to baseline (control) synaptic function generally indicates that neuronal damage has occurred, as further exemplified in Example 4.

Compounds capable of inducing neurodegeneration may include excitotoxic molecules such as glutamate receptor agonists. Examples of glutamate receptor agonists include N-methyl-D-aspartate (NMDA) and amino-3-hydroxy-5-methyl-4-isoxazolepropionic acid (AMPA).

Other compounds capable of inducing neurodegeneration may include oxidative compounds that produce oxidative damage. As used herein, “oxidative damage” refers to damage to neuronal cells or tissue caused by an oxidative compound. The term “oxidative compound” generally refers to compounds having the capability to oxidize substances. Examples of oxidative compounds that may induce oxidative damage include, but are not limited to, reactive oxygen species such as hydrogen peroxide (H₂O₂), superoxide radical, hydroxyl radical, nitric oxide, ozone, thiyl radicals, and carbon-centered radicals (e.g., trichloromethyl radical).

In another variation, the method detects candidate compounds that are capable of protecting against neuronal damage (neuroprotectants or neuroprotective compounds). After baseline synaptic transmission is recorded, a candidate compound is added to the culture medium such that it contacts the sample. Synaptic transmission is then recorded at various timepoints, e.g., at 3 hours, 1 day, 2 days, or 3 days or more, after introduction of the candidate compound, depending on investigator preference. An increase in synaptic transmission observed when compared to baseline (control) synaptic function generally indicates that protection against neuronal damage has occurred, as further exemplified in Examples 5-7.

Candidate compounds capable of protecting against neuronal damage may include glutamate receptor antagonists and antioxidants. Examples of glutamate receptor antagonists include MK-801 and memantine. Vitamin E, Vitamin C, and glutathione may be used as antioxidants.

In a further variation, the method detects a culturing condition that induces neuronal damage. Typically, the culturing condition used to obtain baseline synaptic function is altered by manipulations such as temperature or pH changes of the culture medium, introducing a source of a neurodegeneration-inducing compound, exposing the neuronal sample to radiation, or depriving the sample of oxygen or other factors necessary to maintain sample viability. In yet a further variation, the method detects a culturing condition that protects against neuronal damage. Protective culturing conditions may also include specific temperature or pH ranges of culture medium, the introduction of a source of a neuroprotective compound, or exposure to radiation.

In another variation, it may be preferable to use a neuronal sample with a pre-existing defect to detect neuroprotectants. For example, the defect may be genetically induced, as seen in genetically modified animals. The defect may also be mechanically created such that a physical lesion is produced in the neuronal sample.

### EXAMPLES

The following examples serve to more fully describe the manner of using the invention herein described. It is understood that these examples in no way serve to limit the scope of this invention, but rather are presented for illustrative purposes.

### EXAMPLE 1

### Preparation of the MED Probe

Before use, the MED probes (Panasonic, model MED-P530AP, each electrode: 50×50/m, interpolar distance: 300 μm) were soaked in 70% ethanol for 15 minutes, dried, and then sterilized with UV radiation for 15 minutes. The probe surfaces were treated overnight at room temperature with 0.1% polyethylenimine and 25 mM borate buffer, pH 8.4. The probe surface was then dried and rinsed 3 times with sterile distilled water. Finally, the probes were filled with culture medium and stored in a CO₂ incubator until use (for at least one hour). The culture medium was a 2:1 mixture of Basal Medium Eagle Medium (Sigma, Catalog No. B9638) and Earle Balanced Salts Solution (Sigma, Catalog No. E7510), supplemented with the following compounds (in mM): NaCl (20), NaHCO₃ (5), CaCl₂ (0.2), MgSO₄ (1.7), glucose (48), HEPES (26.7); 5% horse serum (GIBCO, Catalog No. 26050) and 10 ml/L penicillin-streptomycin (GIBCO, Catalog No. 10378). The pH of culture medium was then adjusted to 7.2.

### EXAMPLE 2

### Preparation of Neuronal Samples

All procedures for culture preparation were carried out under a sterilized bench. Eleven day-old Sprague-Dawley rats were first sterilized with 70% ethanol, sacrificed by decapitation following anesthesia and the whole brain removed. The brains were immediately soaked in sterile ice-cold MEM (pH 7.2; GIBCO, Catalog No. 61100), supplemented with HEPES (25 mM), Tris-base (10 mM), glucose (10 mM) and MgCl₂ (3 mM). Appropriate portions of the brain were then trimmed by hand and the remaining brain block placed on an ice-cold stage of a vibrating tissue slicer (Leica, model VT1000S). The thickness of the slices was set at 200 μm. The slices were gently taken off from the blade with a pipette. Each slice was trimmed, placed on the center of the MED probe, which was previously coated as mentioned above, and positioned to cover the 8×8 microelectrode array.

After positioning the section on the MED probe, the cutting solution was removed and culture medium added to the slice up to an interface level (approximately 25011). Sterile distilled water was added around the probe to increase humidity and prevent over-drying of the culture medium in the MED probe. The slices on the MED probes were then stored in a CO₂ incubator at 34° C. The medium was exchanged with half volume every day.

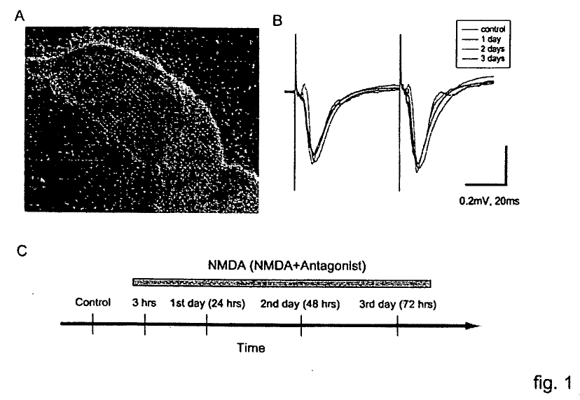

FIG. 1A shows a hippocampal slice cultured on an MED probe for 1 week as well as the positions of the electrodes. Under the experimental conditions, no obvious morphological changes were noticed during at least 2 weeks in culture on the MED probes. Slight to moderate migration of the cells out of the slice was observed, normally starting after 14-20 days. Some flattening of the slices also occurred after two weeks in vitro, which did not interfere with evoked field excitatory postsynaptic potential (fEPSP) recordings.

During the first seven days of in vitro culturing, some increase in the amplitude of fEPSPs was observed; however, after 7-10 days, the responses stabilized and remained stable over subsequent recording periods. These results are consistent with the findings of Muller et al. (Dev. Brain Res., 71:93-100 (1993)). As a result, all experiments described herein were done after at least 10 days of culturing slices on the MED probes.

### EXAMPLE 3

### Baseline Electrophysiological Recordings

For baseline electrophysiological recordings in hippocampal samples, the MED probes containing the samples were removed from the incubator and placed in a smaller CO₂ incubator at 34° C. and connected to the stimulation/recording component of the probe. The culture medium was replaced with sterile artificial cerebrospinal fluid (ACSF) of the following composition (in mM): NaCl (124), NaHCO₃ (26), glucose (10), KCl (3), NaH₂PO₄ (1.25), CaCl₂ (2), MgSO₄ (1), and HEPES (10).

Evoked field potentials at all 64 sites were then recorded simultaneously with the multi-channel recording system (Panasonic, MED64 system) at a 20 kHz sampling rate. One of the planar microelectrodes of the 64 available was used for cathode stimulation. Bipolar constant current pulses (10 to 45 μA, 0.1 msec) were then produced. To collect typical responses in field CA1, one of the electrodes in the Schaffer collateral fibers was selected as a stimulating electrode while another one in stratum radiatum was selected as a recording electrode. Synaptic responses were recorded at eight-step stimulation intensities (10 to 45 μA, 5 μA steps). After each recording session, ACSF was replaced with culture medium and the samples in the probes were returned to the CO₂ incubator.

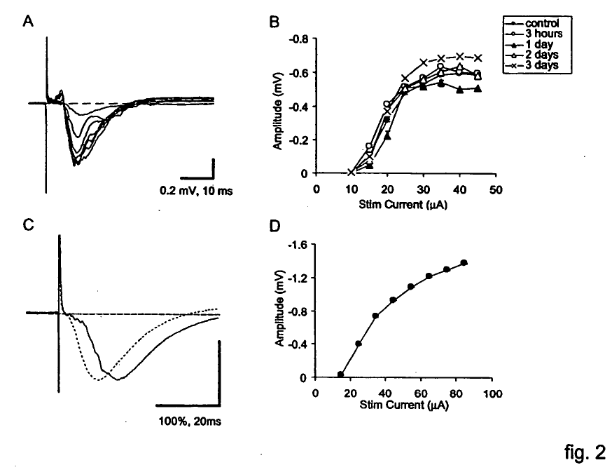

fEPSPs were recorded at 20 s intervals using a paired-pulse stimulation paradigm with an inter-pulse interval of 50 msec. As shown in FIG. 1B, paired pulse facilitation was typically observed. Slope and decay time of the responses were remarkably similar to those recorded in acutely prepared slices using the same system (FIG. 2C). However; a delay after the stimulus artifact was generally observed in the culture conditions, possibly due to the lack of myelination of axons in cultured slices as previously reported (Buchs et al., Dev. Brain Res., 71:81-91 (1993)).

In order to obtain a better evaluation of the effects produced by tested compounds, control (baseline) experiments were carried out in parallel with those including test compounds. In all experiments, the amplitude of fEPSPs in control samples was taken as being the amplitude generated from healthy tissue. Various stimulation currents (ranging from 10 to 45 μA) were used to obtain corresponding fEPSPs (FIG. 2A). Amplitudes measured at the peaks of fEPSPs were plotted as a function of the stimulation currents thus providing input-output (I/O) curves (FIG. 2B), which were also very similar to those obtained in acute hippocampal slices (FIG. 2D).

### EXAMPLE 4

### Neurodegeneration Induced By NMDA and AMPA

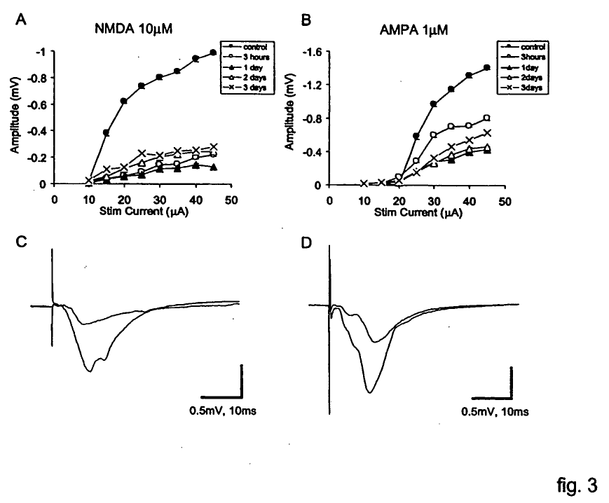

In general, incubation of cultured hippocampal samples chronically exposed to either NMDA or AMPA resulted in dose-dependent decreases in synaptic responses. Representative I/O relationships and FEPSP recordings before and at various times after incubation in the presence of NMDA (10 μM) or AMPA (1 μM) are shown in FIG. 3.

After three hours of incubation in the presence of 10 μM NMDA, the maximal amplitude of synaptic responses was reduced to 18±4% (n=3) of control values and this decrease did not change significantly during subsequent incubation periods (FIG. 3A, representative recordings are shown in FIG. 3C).

When 1 μM AMPA was applied for three hours, the decrease in synaptic response was smaller, and the responses continued to decrease at subsequent time points, stabilizing after 1 day (FIG. 3B, representative recordings are shown in FIG. 3D).

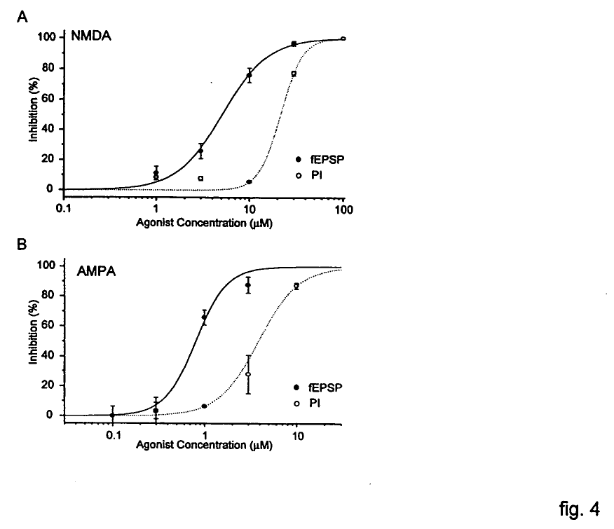

The mechanisms underlying the reduction in amplitude of synaptic responses produced by incubation with these two excitotoxins were further studied by applying various concentrations of NMDA and AMPA to tissue samples. As in the control experiments, the plateau values of the respective I/O curves were used for the analysis (FIGS. 4A and 4B). Fitting the dose-response curves with the Hill function generated EC₅₀ values of 5.2±0.5 μM for NMDA and 0.81±0.1 μM for AMPA, with Hill coefficients of 1.7±0.2 and 2.6±0.8, respectively. For comparison, dose-response curves obtained with the PI uptake method were plotted in the same figures (FIGS. 4A and 4B, open circles and dashed lines). EC₅₀ values from the PI uptake method were 21.6±3.4 μM for NMDA and 3.87±0.1 μM for AMPA and Hill coefficients were 3.7±1.0 and 2.0±0.1, respectively. The dose-response curves obtained with electrophysiology were shifted toward the left and were also generally less steep (at least for NMDA) than those observed with the PI method, suggesting that these two methods reveal different cellular mechanisms activated by NMDA and AMPA.

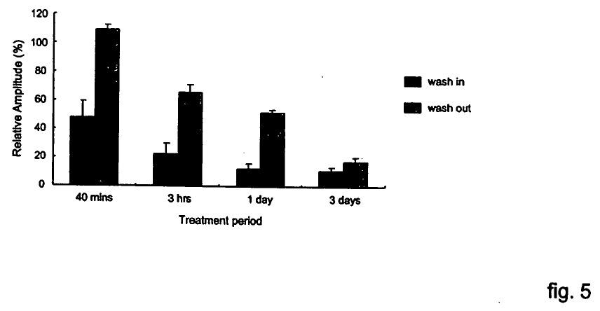

To further address this question, the time-course for recovery of synaptic responses after agonist removal was investigated. At the end of incubation in the presence of 10 μM NMDA for 40 min., 3 hours, 1 day, and 3 days, the amplitudes of synaptic responses were 48±12%, 22±7%, 13±3%, and 11±3% of control values, respectively (n=3). However, as shown in FIG. 5, following one hour of NMDA wash-out under the same conditions, synaptic responses were 109±4%, 66±5%, 52±2%, and 18±3% of the initial amplitude, respectively, indicating that synaptic responses in fact gradually decrease and become irreversibly diminished only after 3 days of continuous treatment with NMDA. Similar results were obtained for AMPA: a 40 min. incubation in the presence of 3 μM AMPA led to a large decrease in the amplitude of synaptic responses (12±5% of control values) and the responses recovered to up to 93±2% of control after AMPA was removed for 1 hour (n=3); 3 days of incubation in the presence of 1 μM AMPA decreased synaptic response amplitudes to 46±4% (n=3) and no recovery was observed even after 24 hours of AMPA removal (results not shown).

Thus, partial recovery of synaptic responses at earlier time-points in our experiments suggests that it is unlikely that only neuronal degeneration contributes to decreases in synaptic transmission. Additional suppression of synaptic transmission may occur due to agonist-induced opening of glutamate receptor channels and subsequent neuronal depolarization. Furthermore, this data is useful because it allows identification of a window in which neuronal resuscitation may be possible.

### EXAMPLE 5

### Method For Detecting Neuroprotectants

An assay for identifying neuroprotectants first involves testing the stability of the recordings by comparing FEPSP amplitudes measured at the beginning of the experiments with those measured later, e.g., after at least 3, 24, 48, and 72 hours at the same stimulation intensity (FIG. 1C). For data analysis, the amplitude of the maximal (plateau) value of the I/O curve is used to determine stability of the recordings. Typically, it corresponds to responses obtained by stimulation intensities of 3040 μA (FIG. 2B). Recordings obtained from 23 control experiments indicated that the relative amplitude of fEPSP after 3 days of chronic recording was 107±4% (n=23) of the control value, indicating a slight increase in the amplitude over the period of measurements (10-13 days in vitro (DIV), data not shown).

For longer incubation periods (i.e., 20 days DIV), the response amplitude was found to be 107±4% of control values (n=2). In one sample, recordings were even possible after 45 DIV, although with approximately a 50% reduction in amplitude. However, in the present study all results are presented for samples cultured no longer than 20 days DIV.

### EXAMPLE 6

### Neuroprotective Effect of MK-801

Example 6 demonstrates that MK-801 is a neuroprotectant. NMDA was selected as the excitotoxin. Based on the dose-response analysis described above (FIG. 4A), 10 μM NMDA was chosen as the concentration of agonist for use in this neuroprotectant assay. Since we were interested in evaluating the neuroprotective effects of MK-801 on neuronal samples chronically exposed to NMDA, we did not analyze results obtained, if any, within the first 3 hours of the assay. The neuronal samples and MED probes were prepared as described above.

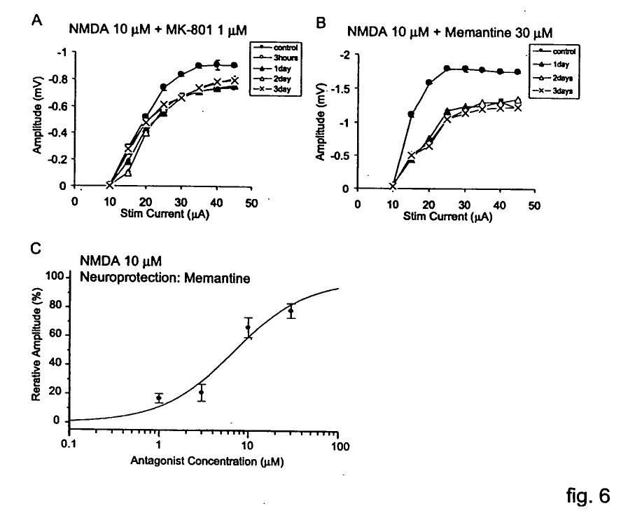

After performing control measurements, neuronal samples were incubated in culture medium containing 10 μM NMDA and 1 μM MK-801 for various periods of time. Under these conditions, synaptic responses were only slightly depressed from 3 hours up to 3 days of incubation (FIG. 6A), indicating that 1 μM MK-801 almost completely protected synaptic transmission against NMDA-mediated neurotoxicity, a result consistent with previous studies using different markers of synaptic damage (Peterson et al., 1989; Pringle et al.; Kristensen et al., 2001). The relative amplitude of synaptic responses measured at stimulation intensities corresponding to the plateau of I/O curves after 3 days of incubation was 91±6% of control (n=3).

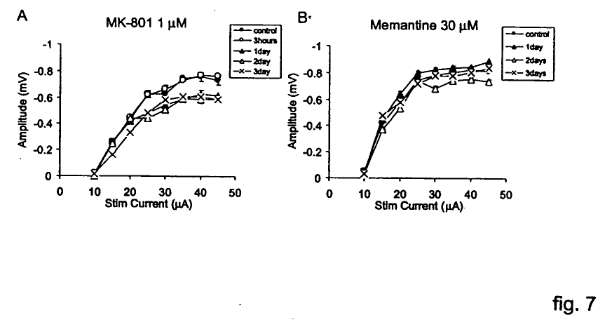

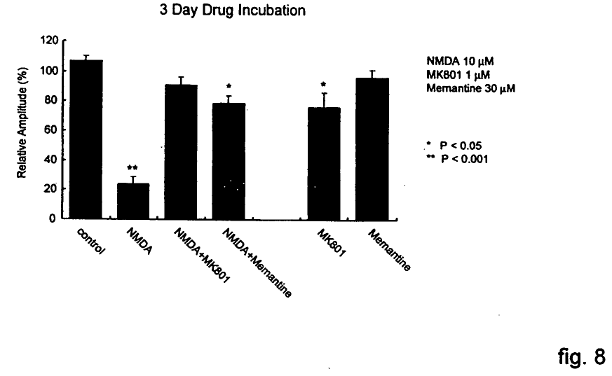

Furthermore, incubation of the neuronal samples in the presence of 1 μM MK-801 alone resulted in decreased synaptic responses after 1 day. Interestingly, synaptic responses were not affected after 3 hours of incubation (FIG. 7A). However, after 1, 2, and 3 days of incubation, the amplitudes of responses were decreased to 83±6%, 79±11%, and 76±10% (n=3) of control, respectively. These decreases in amplitude were not significantly different from the decrease in amplitudes produced by 1 μM MK-801 in the presence of 10 μM NMDA (FIG. 6A).

### EXAMPLE 7

### Neuroprotective Effect of Memantine

Example 7 demonstrates that memantine is a neuroprotectant. NMDA was selected as the excitotoxin. Based on the dose-response analysis described above (FIG. 4A), 10 μM NMDA was chosen as the concentration of agonist for use in this neuroprotectant assay. Since we were interested in evaluating the neuroprotective effects of memantine on tissue samples chronically exposed to NMDA, we did not analyze results obtained, if any, within the first 3 hours of the assay. The neuronal samples and MED probes were prepared as described above.

After performing control measurements, neuronal samples were incubated in culture medium containing 10 μM NMDA and 30 μM memantine for various periods of time. As shown in FIG. 6B, the pattern of protection was found to be relatively similar to that of MK-801, as synaptic responses were significantly protected for up to 3 days of incubation. However the degree of protection was much smaller than that provided by MK-801, as only 78±5% (n=3) of the initial amplitude was preserved under these conditions. The concentration dependency of memantine protection was studied using 10 μM NMDA and various concentrations of memantine. The dose-response curve was fitted with the Hill equation and provided IC₅₀ values of 6.9±1.6 μM, with a Hill coefficient of 1.1±3, as shown in FIG. 6C.

In contrast to MK-801, long-term incubation of neuronal samples in the presence of memantine alone, even at 30 μM, did not result in a significant decrease in synaptic transmission (FIG. 7B).

### EXAMPLE 8

### Neurodegeneration Induced by Ischemia

Preparation of neuronal samples and measurement of baseline electrophysiological recordings are performed as described in Examples 2 and 3, respectively. Samples are washed in recording solution containing: 126 mM NaCl; 3 mM KCl; 1.25 mM NaH₂PO₄; 24 mM NaHCO₃; 1 mM MgSO₄; 10 mM HEPES; 10 mM D-glucose; and 2 mM CaCl₂. pH is then adjusted to about 7.3. The sample is then placed in an air-tight plastic chamber and exposed to an atmosphere composed entirely of argon. Slices are incubated in the chamber for approximately 60 to 90 minutes. Synaptic transmission is then measured as described in Example 4.

Although this example uses an atmosphere composed entirely of argon to induce tissue ischemia, other noble gases such as krypton, xenon, and radon, or other suitable oxygen-free gases, may also be used.

### EXAMPLE 9

### Neurodegeneration Induced by Oxidative Damage

Preparation of neuronal samples and measurement of baseline electrophysiological recordings are performed as described in Examples 2 and 3, respectively.

1 mM hydrogen peroxide (H₂O₂) is then added to the culture medium at concentrations between about 25 μM and 75 μM. The samples are incubated with the H₂O₂ for about one hour. Synaptic transmission is then measured 24 hours later, similar to the protocol described in Example 4.

All publications, patents, and patent applications cited herein are hereby incorporated by reference in their entirety for all purposes to the same extent as if each individual publication, patent, or patent application were specifically and individually indicated to be so incorporated by reference. Although the foregoing invention has been described in some detail by way of illustration and example for purposes of clarity of understanding, it will be readily apparent to those of ordinary skill in the art in light of the teachings of this invention that certain modifications may be made thereto without departing from the spirit or scope of the appended claims.

1. A method for detecting compounds that induce changes in synaptic transmission between neurons or induce neurodegeneration in vitro comprising:
a) providing a device having a plurality of microelectrodes on a substrate configured to contact a neuronal sample and apply an electric stimulus to the neuronal sample;
b) contacting the neuronal sample with the plurality of microelectrodes;
c) measuring a baseline synaptic transmission of the neuronal sample;
d) contacting the neuronal sample with a first candidate compound;
e) measuring a first resultant synaptic transmission of the neuronal sample at one or more timepoints after step (d); and
f) comparing the first resultant synaptic transmission with the baseline synaptic transmission,
wherein a decrease in synaptic transmission between the first and baseline synaptic transmissions indicates that the candidate compound induces neurodegeneration in the neuronal sample.

2. The method of claim 1 wherein the first candidate compound comprises an excitotoxic molecule.

3. The method of claim 2 wherein the excitotoxic molecule comprises a glutamate receptor agonist.

4. The method of claim 3 wherein the glutamate receptor agonist comprises N-methyl-D-aspartate (NMDA).

5. The method of claim 3 wherein the glutamate receptor agonist comprises c-amino-3-hydroxy-5-methyl-4-isoxazolepropionic acid (AMPA).

6. The method of claim 1 wherein the first candidate compound induces oxidative damage in the neuronal sample.

7. The method of claim 6 wherein the first candidate compound comprises hydrogen peroxide.

8. The method of claim 1 further comprising the steps of:
g) contacting the neuronal sample with a second candidate compound;
h) measuring a second resultant synaptic transmission of the neuronal sample at one or more timepoints after step (g);
i) comparing the second resultant synaptic transmission with the first synaptic transmission,
wherein an increase in synaptic transmission between the second and first synaptic transmissions indicates that the candidate compound protects against neurodegeneration in the neuronal sample.

9. The method of claim 8 wherein the second candidate compound comprises a glutamate receptor antagonist.

10. The method of claim 9 wherein the glutamate receptor antagonist comprises MK-801.

11. The method of claim 9 wherein the glutamate receptor antagonist comprises memantine.

12. The method of claim 8 wherein the second candidate compound comprises an antioxidant.

13. The method of claim 12 wherein the antioxidant comprises vitamin E.

14. The method of claim 8 wherein the first and second candidate compounds contact the neuronal sample simultaneously.

15. The method of claim 8 wherein the first and second candidate compounds contact the neuronal sample sequentially.

16. The method of any one of claims 1-15 wherein one or more of the measuring steps comprises measuring amplitude of electrical waveforms generated by the neuronal sample.

17. A method for detecting a culturing condition that induces changes in synaptic transmission between neurons or induces neurodegeneration in vitro comprising:
a) providing a device having a plurality of microelectrodes on a substrate configured to contact a neuronal sample and apply an electric stimulus to the neuronal sample, and a culturing chamber that provides a first culturing condition;
b) contacting the neuronal sample with the plurality of microelectrodes;
c) measuring a baseline synaptic transmission of the neuronal sample;
d) altering the first culturing condition to produce a second culturing condition;
e) measuring a first synaptic transmission of the neuronal sample at one or more timepoints after step (d); and
f) comparing the first synaptic transmission with the baseline synaptic transmission,
wherein a decrease in synaptic transmission between the first and baseline synaptic transmissions indicates that the second culturing condition induces neurodegeneration in the neuronal sample.

18. The method of claim 17 wherein the step of altering the first culturing condition comprises exposing the neuronal sample to an ischemia-inducing condition.

19. The method of claim 18 wherein the neuronal sample is exposed to an atmosphere composed entirely of a noble gas.

20. The method of claim 18 wherein the neuronal sample is exposed to an atmosphere composed entirely of argon.

21. The method of claim 17 wherein the step of altering the first culturing condition comprises exposing the neuronal sample to radiation.

22. The method of claim 17 wherein the step of altering the first culturing condition comprises exposing the neuronal sample to a temperature change.

23. The method of claim 17 further comprising the steps of:
g) altering the second culturing condition;
h) measuring a second resultant synaptic transmission of the neuronal sample at one or more timepoints after step (g);
i) comparing the second resultant synaptic transmission with the first synaptic transmission,
wherein an increase in synaptic transmission between the second and first synaptic transmissions indicates that the altered second culturing condition protects against neurodegeneration in the neuronal sample.

24. The method of claim 17 wherein the first and second culturing conditions are altered simultaneously.

25. The method of claim 17 wherein the first and second culturing conditions are altered sequentially.

26. The method of any one of claims 17-25 wherein one or more of the measuring steps comprises measuring amplitude of electrical waveforms generated by the neuronal sample.

27. A system for assessing changes in synaptic transmission between neurons or changes in neurodegeneration in vitro in a neuronal sample, comprising:
a) a device comprising a plurality of microelectrodes on a substrate configured to contact a neuronal sample and apply an electric stimulus to the neuronal sample, and a culturing chamber that provides a culturing condition; and
b) a source of a neurodegeneration-inducing compound;
wherein the device is adapted to contact the neuronal sample with the plurality of microelectrodes and to assess changes in synaptic transmission by comparing the synaptic transmission of the neuronal sample at one or more timepoints after exposure of the neuronal sample to the neurodegeneration-inducing compound or a culturing condition that induces neurodegeneration.

28. The system of claim 27 wherein the neurodegeneration-inducing compound comprises an excitotoxic molecule.

29. The system of claim 28 wherein the excitotoxic molecule comprises a glutamate receptor agonist.

30. The system of claim 29 wherein the glutamate receptor agonist comprises N-methyl-D-aspartate (NMDA).

31. The system of claim 29 wherein the glutamate receptor agonist comprises α-amino-3-hydroxy-5-methyl-4-isoxazolepropionic acid (AMPA).

32. The system of claim 27 wherein the compound comprises an oxidative compound.

33. The system of claim 32 wherein the oxidative compound comprises hydrogen peroxide.

34. The system of claim 27 wherein the culturing condition is an ischemia-inducing condition.

35. The system of claim 34 wherein the neuronal sample is exposed to an atmosphere composed entirely of a noble gas.

36. The system of claim 34 wherein the neuronal sample is exposed to an atmosphere composed entirely of argon.

37. The system of claim 27 wherein the neuronal sample is exposed to radiation.

38. The system of claim 27 wherein the neuronal sample is exposed to a temperature change.

39. The system of claim 27 wherein the device is adapted to measure a baseline synaptic transmission of the neuronal sample prior to exposure of the neuronal sample to the neurodegeneration-inducing compound.

40. A system for assessing changes in synaptic transmission between neurons or changes in neurodegeneration in vitro in a neuronal sample, comprising:
a) a device comprising a plurality of microelectrodes on a substrate configured to contact a neuronal sample and apply an electric stimulus to the neuronal sample, and a culturing chamber that provides a culturing condition; and
b) a source of a neuroprotective compound;
wherein the device is adapted to contact the neuronal sample with the plurality of microelectrodes and to assess changes in synaptic transmission by comparing the synaptic transmission of the neuronal sample at one or more timepoints after exposure of the neuronal sample to the neuroprotective compound or a culturing condition that protects against neurodegeneration.

41. The system of claim 40 wherein the compound comprises a glutamate receptor antagonist.

42. The system of claim 41 wherein the glutamate receptor antagonist comprises MK-801.

43. The system of claim 42 wherein the glutamate receptor antagonist comprises memantine.

44. The system of claim 40 wherein the compound comprises an antioxidant.

45. The system of claim 44 wherein the antioxidant comprises vitamin E.

46. The system of claim 40 wherein the device is adapted to measure a baseline synaptic transmission of the neuronal sample prior to exposure of the neuronal sample to the neuroprotective compound
